(19) United States
(12) Patent Application Publication
(10) Pub. No.: US 2026/0098861 A1
(43) Pub. Date: Apr. 9, 2026

(54) ENGINEERED PHAGE AND KIT FOR CAPTURING SARS-COV-2 AND METHOD FOR DETECTING SARS-COV-2 VIRUS BY MEANS OF SAID PHAGE OR KIT

(71) Applicants: ALMA MATER STUDIORUM - UNIVERSITA’ DI BOLOGNA, Bologna (BO) (IT); UNIVERSITA’ DEGLI STUDI DI MESSINA, MESSINA (ME) (IT)

(72) Inventors: Salvatore GUGLIELMINO, CATANIA (CT) (IT); Maria Giovanna RIZZO, SANTA FLAVIA (PA) (IT); Nicoletta PALERMO, ENNA (EN) (IT); Marco Sebastiano NICOLO', REGGIO DI CALABRIA (RC) (IT); Emanuele Luigi SCIUTO, CATANIA (CT) (IT); Matteo CALVARESI, BOLOGNA (BO) (IT); Alberto DANIELLI, SAN LAZZARO DI S. (BO) (IT); Luca PRODI, BOLOGNA (BO) (IT); Sabrina CONOCI, TREMESTIERI ETNEO (CT) (IT)

(21) Appl. No.: 19/116,251

(22) PCT Filed: Sep. 28, 2023
(86) PCT No.: PCT/IB2023/059704

§ 371 Date: Mar. 27, 2025

(30) Foreign Application Priority Data
Sep. 30, 2022 (IT) 102022000020199

(51) Int. Cl. G01N 33/569; C12N 7/00; G01N 33/543

(52) U.S. Cl. CPC G01N 33/56983; C12N 7/00; G01N 33/54326; C12N 2795/14121; G01N 2333/165

(57) ABSTRACT

A phage for the specific capture of the SARS-CoV-2 virus, said phage being an M13 phage engineered to display on the P8 protein of its coat either FHKGGYEKTWKLGD sequence peptides or EFTSKAR sequence peptides, said peptides having specific affinity for the Spike S1 protein of the SARS-CoV-2 virus.

### CROSS-REFERENCE TO RELATED APPLICATIONS

This application is a U.S. National Phase application under 35 U.S.C. 371 of International Application No. PCT/IB2023/059704, filed on Sep. 28, 2023, which claims priority to Italian Patent Application 102022000020199, filed on Sep. 30, 2022. The entire disclosures of the above applications are incorporated herein by reference.

### SEQUENCE LISTING

This application contains references to amino acid sequences and/or nucleic acid sequences which have been submitted concurrently herewith as the sequence listing file entitled “000042usnp_SequenceListing.XML”, file size 3,661 bytes, created on Mar. 21, 2025. The aforementioned sequence listing is hereby incorporated by reference in its entirety pursuant to 37 C.F.R. § 1.52(e)(5).

### FIELD

The present invention relates to the technical fields of biomedicine and molecular biology, and relates, in particular, to an engineered phage for the capture of the SARS-CoV-2 virus.

### BACKGROUND

This section provides background information related to the present disclosure, which is not necessarily prior art.

The new coronavirus of 2019, called SARS-CoV-2, which causes the respiratory disease called COVID-19, has spread rapidly on a global scale.

Compared to the SARS-CoV and MERS-CoV coronaviruses, which have caused previous outbreaks of respiratory syndrome, the novel SARS-CoV-2 virus is highly contagious with high transmissibility even in patients with mild symptoms. COVID-19 disease caused by the virus has spread rapidly on a global scale and there is therefore an urgent need for effective and rapid diagnosis to detect SARS-CoV-2 infection. The outer surface of the SARS-CoV-2 virus is covered with spike proteins, which represent one of the peculiar characteristics of the coronavirus family.

The spike protein of SARS-CoV-2 is the main mechanism that the virus uses to infect target cells. This protein is made up of two main components: the S1 subunit and the S2 subunit. The S1 subunit is a very flexible region and contains the domain called RBD, through which the virus is able to recognize and bind the ACE2 receptor, which is the gateway of the virus into the cells of the human body.

Some antibodies specific to SARS-Cov-2 are currently known and described in the following documents.

CN113861288A describes a broad-spectrum neutralizing antibody to coronavirus SARS-CoV-2. Phage antibody library technology is used, successfully obtaining a broad spectrum neutralizing single domain antibody B3A3 and I3A10 specifically combined with SARS-CoV-2 spike RBD protein. The single domain antibody of the disclosed invention has a high affinity for the antigen and has a clear inhibitory effect against the major epidemic strain SARS-CoV-2. The document also illustrates the use of magnetic beads.

This document describes the use of the phage display methodology to generate antibodies (nanobodies) with antiviral activity against SARS-CoV-2 for therapeutic purposes. In the present invention, phages are instead used for diagnostic and non-therapeutic purposes, in that, as specified below, such phages display the peptide sequence FHKGGYEKTWKLGD on the major phage coat protein (P8) with specific affinity for the viral Spike protein to capture and concentrate SARS-CoV-2 directly in biological samples for diagnostic purposes.

The CN113444170A document describes a phage display antibody library and a filter antibody capable of combining with the S protein of the novel coronavirus SARS-CoV-2; the invention is based on synthetic biology and phage display technology; introducing the mutation into the hypervariable region of the antibody variable region; and transferring the gene to the Coli bacillus, so as to construct a synthetic antibody library containing 108 antibodies; the phage display antibody library of the invention can filter the antibody with specificity and detection function, expanding the powerful resource of biological research and medical diagnosis.

The known document describes the use of a methodology based on the generation of antibody libraries for a selective recognition of the Sars-Cov-2 S1 protein. This known document, although it suggests the diagnosis of Sars.Cov-2 by antibodies that recognize the virus, is nevertheless very different from the present invention since:
the system according to the invention uses peptide sequences displayed on the major phage coat protein (P8) present in about 2700 copies (while the aforementioned known patent uses the phage coat P3);
the entire phage structure is used for the functionalization of the magnetic beads for the capture and concentration of the Sars-Cov-2 virus, which is missing in the known patent.

Also known is the CN111333722A document which describes SARS-CoV-2 inhibitors and their application, in particular a method for SARS-CoV-2 neutralizing antibodies and their application. The document uses phage display technology to build a library of high-capacity human phage immune antibodies and the SARS-CoV-2-S protein as a drone, filter and single-chain human antibody fragment, obtaining the antibody with a strong effect on the SARS-CoV-2 virus. The antibody of the invention can be used for the treatment of diseases caused by novel coronavirus infections and has important clinical application value.

Still, CN111592595A describes neutralizing antibody of the novel coronavirus SARS-Cov-2 and its application. The antibody may be used for the preparation of diagnostic reagents or diagnostic kits, drugs or pharmaceutical compositions for the detection, prevention and treatment of COVID-19. The invention uses phage display technology to target SARS-Cov-2-RBD and SARS-Cov-1-RBD for differential antibody screening, obtaining a neutralizing antibody against the novel coronavirus SARS-Cov-2.

The main difference between these documents and the present invention, is that in the latter phages are used for diagnostic and non-therapeutic purposes, as, as specified below, such phages display the peptide sequence FHKGGYEKTWKLGD on the major phage coat protein (P8) with specific affinity to the viral spike protein to capture and concentrate SARS-CoV-2 directly in biological samples for diagnostic purposes.

The CN111647054A document describes a reagent for the detection of the antibody of the novel coronavirus SARS-CoV-2 and its application, in particular it relates to a polypeptide, the sequence of which comprises a novel coronavirus SARS-CoV-2 corresponding to SEQ ID NO.1 A primer composition for the synthesis of the polypeptide, the method of preparation of the polypeptide, the polypeptide for the preparation of reagents for the detection or diagnosis of the novel coronavirus SARS-CoV-2, in particular it can be used for the preparation of strips of reagents in colloidal gold and the relative kit. The specific primer designed by the invention can successfully synthesize the S protein/N protein antigen peptide with excellent binding activity, and the colloidal gold chromatography reagent strip prepared therefrom can quickly and effectively detect the anti-S protein/N protein antibody of the novel coronavirus SARS-CoV-2 and prevent the generation of false negative results. The document describes the use of the phage display technique.

However, what is described in the cited document refers to a diagnostic system for the detection of IgG/IgM antibodies against coronavirus and not to the direct diagnosis of Sars-Cov-2 virus, as in the present invention.

### SUMMARY

This section provides a general summary of the disclosure, and is not a comprehensive disclosure of its full scope or all of its features.

The object of the invention is to provide a method of detecting SARS-CoV-2 virus.

This and other objects are achieved with an engineered phage engineered for the capture of the SARS-CoV-2 virus as claimed in the appended claims.

The phage for specific capture of SARS-CoV-2 virus according to the invention is an engineered M13 phage displaying on the major coat protein of the phage, i.e. P8 protein, peptides of sequence FHKGGYEKTWKLGD (hereinafter referred to as capture phage S-α) or EFTSKAR (hereinafter referred to as capture phage S1-6), both with specific affinity for SARS-CoV-2 virus Spike S1 protein.

The kit for capturing SARS-CoV-2 virus according to the invention comprises a surface functionalized with S-α capture phages or with FHKGGYEKTWKLGD peptides or with S1-6 capture phages or with EFTSKAR peptides. The surface is preferably chosen from magnetic beads, metallic materials (e.g. gold or platinum), semiconductors (e.g. silicon or silicon nitride) or polymers (e.g. nitrocellulose).

The method for obtaining the aforementioned capture phages S-α with the FHKGGYEKTWKLGD peptide, or the aforementioned capture phages S1-6 with the EFTSKAR peptide according to the invention provides for the engineering, by means of the phage display technique, of M13 phages.

More specifically, the method includes the steps of:
providing a phage display library of M13 phages,
biopanning the phage library using His-tagged S1 spike proteins by:
   providing magnetic microspheres functionalized with SARS-CoV-2 Spike S1 proteins,
   or selecting against the aforementioned functionalized magnetic microspheres the phage library previously selected through biopanning, or eluting the selected phages and separating them from the magnetic microspheres,
   or isolating, among the selected phages, phages displaying on the P8 protein of their coat peptides of FHKGGYEKTWKLGD sequence or EFTSKAR sequence.

The detection method of the SARS-CoV-2 virus according to the invention by means of the aforementioned kit containing the S-α capture phages or the FHKGGYEKTWKLGD peptides or the S1-6 capture phages or the EFTSKAR peptides, immobilized on a surface of the aforementioned type, provides for the steps of:
providing a sample containing the SARS-CoV-2 virus,
capturing the SARS-CoV-2 virus using S-α capture phages or FHKGGYEKTWKLGD peptides or S1-6 capture phages or EFTSKAR peptides immobilized on the surface,
isolating the SARS-CoV-2 virus through appropriate washing procedures,
detecting the capture of the virus by the S-α capture phages or the FHKGGYEKTWKLGD peptides or the S1-6 capture phages or the EFTSKAR peptides, by an appropriate transduction method, for example optical or electrical or electrochemical or electro chemiluminescent transduction.

In the case of using magnetic microspheres as a surface, the step of isolating the virus by washing involves the capture of the magnetic microspheres by a magnetic device.

In accordance with the invention, the SARS-CoV-2 virus can be isolated from various types of samples, such as biopsies and swabs, and then used for the diagnosis of SARS-CoV-2 virus disease.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

These and other characteristics and advantages of the present invention will become clear from the following description of preferred embodiments made by way of example and not limitation with the aid of the accompanying figures, in which elements indicated with the same or a similar numerical reference indicate elements having the same or similar functionality and construction and in which:

FIGS. 1a and 1b represent respectively the capture phage S-α with the FHKGGYEKTWKLGD peptide sequence and the S1-6 capture phage with the EFTSKAR peptide sequence;

FIGS. 2a and 2b represent, respectively, a surface functionalized with the FHKGGYEKTWKLGD peptide sequence and a surface functionalized with the EFTSKAR peptide sequence;

FIG. 3 represents a schematic diagram of a possible embodiment of the SARS-CoV-2 virus capture method in the case of surfaces represented by magnetic microspheres functionalized with capture phages.

### DETAILED DESCRIPTION

Example embodiments will now be described more fully with reference to the accompanying drawings.

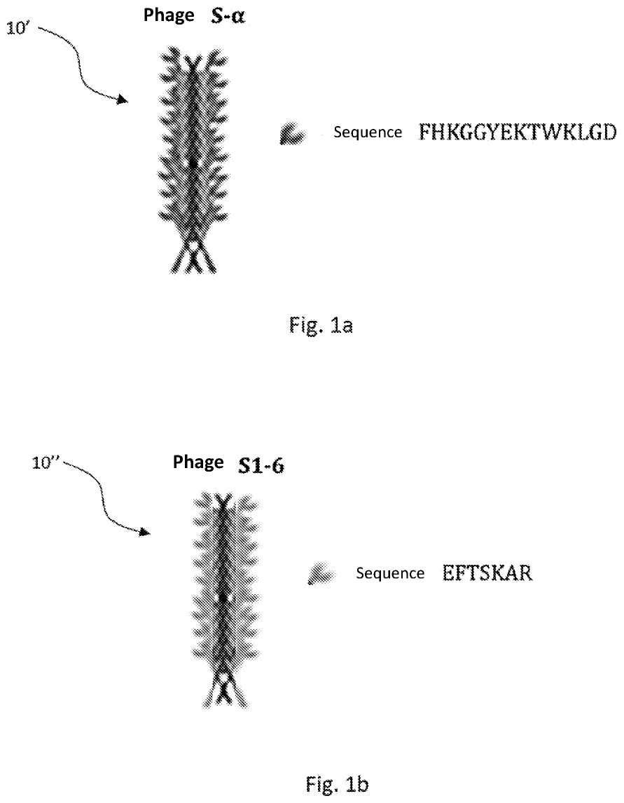

In accordance with the present invention and with reference to FIGS. 1a and 1b, a phage (phage clone) for the specific capture of the SARS-CoV-2 virus is an M13 phage suitably engineered so as to show on the major protein of its coat, i.e. on the P8 protein, hundreds of peptides (up to 2800) of FHKGGYEKTWKLGD or EFTSKAR sequence—In FIGS. 1a and 1b, the aforementioned phages are indicated respectively with reference 10′ and 10″. The FHKGGYEKTWKLGD or EFTSKAR sequence peptides have a specific affinity for the Spike S1 protein of SARS-CoV-2 virus.

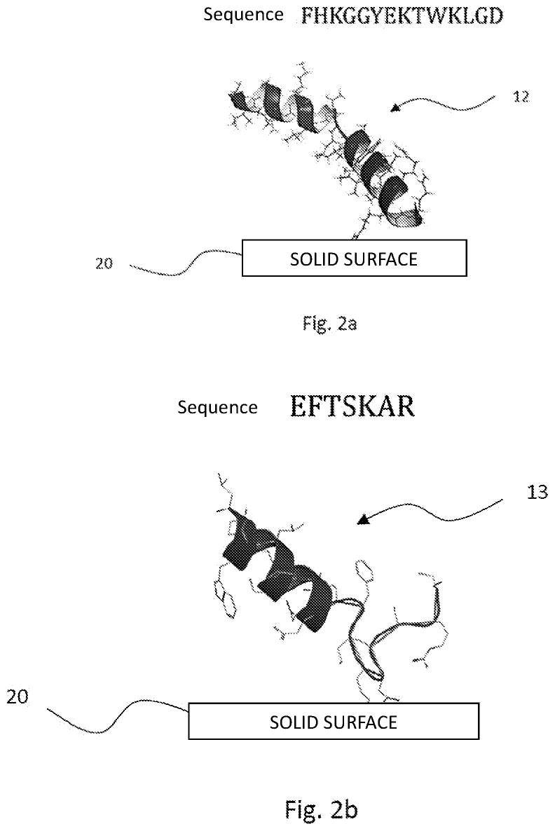

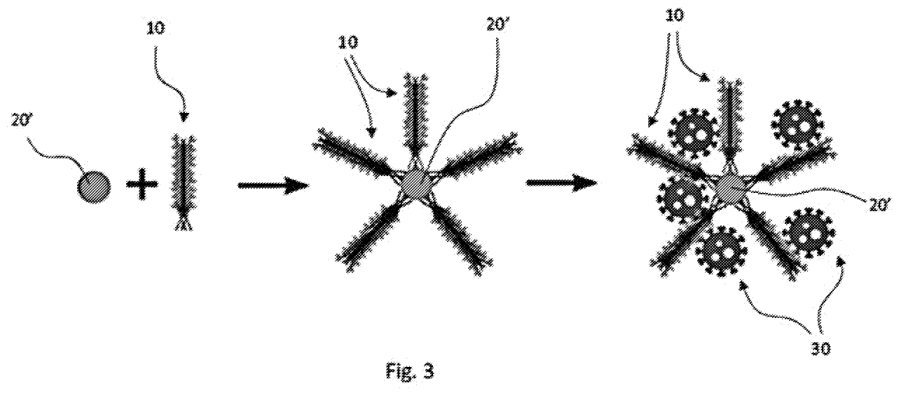

In accordance with the invention, a kit for the capture of SARS-CoV-2 virus comprises a surface functionalized with the capture phages S-α or S1-6 (as shown in FIG. 3) or functionalized with FHKGGYEKTWKLGD peptides or with EFTSKAR peptides (as shown in FIGS. 2a and 2b). In FIGS. 2a and 2b the surface is indicated with reference 20, the FHKGGYEKTWKLGD peptide with reference 12 and the EFTSKAR peptide with reference 13. In FIG. 3 the surface is indicated with reference 20′. The surfaces 20 and 20′ are preferably chosen from magnetic beads, metallic materials (for example in gold or platinum), semiconductors (for example in silicon or silicon nitride) or polymers (for example nitrocellulose). The magnetic microspheres preferably have a diameter of between 0.5 μm and 2.7 μm.

With reference to FIG. 3, a method of capturing the SARS-CoV-2 virus (indicated with reference 30) by means of the aforementioned kit containing the capture phages S-α or S1-6 (indicated with reference 10) and magnetic microspheres (indicated with reference 20′) is described below.

The method involves the steps of:
providing a sample containing the SARS-CoV-2 virus,
capturing the SARS-CoV-2 virus using the S-α or S1-6 capture phages immobilized on the magnetic microspheres,
isolating the SARS-CoV-2 virus using a suitable magnetic device (e.g. a magnet).

An operational example of the capture of the SARS-CoV-2 virus by the magnetic microspheres functionalized with the S-α or S1-6 capture phages is illustrated below, which provides that the S-α capture phages are immobilized on the magnetic microspheres.

In accordance with this example, 20 μL (1 mg) of “Chemicell SIMAG-AMINE” magnetic microspheres of 1 μm in diameter are taken from the mother batch tube and added to 180 μL of sterile u.p. (ultrapure) water to have a concentration of 9×10⁹ microspheres/mL. From the aforementioned u.p. water with microspheres, 20 μL are taken and added to another 180 μL of water to have a concentration of 0.1 mg. The microspheres are then washed once with 500 μL of water for 10 min on a wheel at 8 rpm at room temperature and then collected on a magnet. Two washes are carried out with 1 mL of Buffer MES (0.9 g in 50 mL H₂O u.p. pH 6.0) for 10 minutes on a wheel at 8 rpm at room temperature using the magnetic separator. The microspheres are then resuspended in 250 μL MES EDC Buffer (0.01 g EDC in 250 μL MES Buffer). 10 μL of S-α phages are then taken from a 4×10¹³ solution and added, in a tube, to 90 μL of water to bring the total volume to 100 μL, with a ratio of about 336 phages per single microsphere. The aforementioned tube is then manually shaken for 1 minute and incubated on the wheel for 2 hours. The microspheres are then washed three times with 1 mL of PBS for 5 minutes on a wheel collecting in a magnetic separator. 1.5 mL of blocking buffer (PBS+4% BSA 0.005 g of sodium azide) is then added over 1 hour and 30 minutes on the wheel. The microbeads are then collected on a magnet for 20 minutes, washed once with 500 μL PBS and resuspended in 200 μL PBS.

The ability of the aforementioned magnetic microspheres functionalized with the S-α capture phages to detect the SARS-CoV-2 virus was tested by ELISA method, as illustrated below.

20 μL of S-α phage-functionalized microspheres (336 phages per 1.8×10⁷ microspheres) were added to 100 μL of SARS-CoV-2 (e.g., “Amplirum total SARS CoV-2 control SWAB”) at different dilutions (1:5, 1:50, 1:100, 1:500). 100 μL of PBS was used as a control (K-negative) in place of the virus, in 2 mL tubes. After 1 hour of incubation of the wheeled tubes at 100 rpm, 1 wash was carried out with 100 μL of wash buffer and the microspheres were then collected on a magnet for 5 minutes. Subsequently, 100 μL of anti-spike antibody HRP (“monoclonal mouse anti-SARS-CoV-2 Spike”) diluted 1:200 was added to the tubes, which were then incubated for 1 hour on a wheel at 100 rpm. Five washes were then performed with 100 μL of wash buffer. After the last wash, 100 μL of TMB was added and the reaction was blocked with 100 μL of H₂SO₄. The supernatants were then transferred to a 96-well plate for absorbance reading at 450 nm. The results obtained are shown in the following table:

| | SARS- | SARS- | SARS- | SARS- | |
|---|---|---|---|---|---|
| | CoV-2 | CoV-2 | CoV-2 | CoV-2 | |
| | 1:5 | 1:50 | 1:100 | 1:500 | K-negative |
| Absorbance | 4,127 | 0.358 | 0.132 | 0.067 | 0.048 |

The data reported above indicate that the S-α phage, which has the FHKGGYEKTWKLGD functional sequence motif on the P8 protein, is effective in capturing the SARS-CoV-2 virus up to 1:50 dilution (absorbance value greater than three times the absorbance value for the control sample). In particular, the viral suspension “Amplirum total SARS-CoV-2 control SWAB” used contains about 30000 viral copies/mL. 100 μL of the viral suspension was used in the tests carried out, estimating about 600 virions at a 1:5 dilution, about 60 virions at a 1:50 dilution, about 30 virions at a 1:100 dilution and about 6 virions at a 1:500 dilution. Although currently evaluated with the limits of the ELISA technique, the proposed detection method is able to detect between 30 and 60 virions of SARS-CoV-2, i.e. a value similar to that detected with RT-PCR techniques (500 copies/mL).

An exemplary embodiment of the method for obtaining the capture phage described above is outlined below.

In order to search for specific peptides capable of binding the spike S1 protein of the SARS-CoV-2 virus, a phage display library “M13 P8 phage display 12aa” is selected against magnetic microspheres (e.g. Dynabeads spheres) His-Tag functionalised with the spike S1 protein of the SARS-CoV-2 virus.

In a first step of the selection, the phage display library is selected against non-functionalized magnetic beads, so as to eliminate all phages that may non-specifically bind the materials (thus obtaining a so-called subtractive library).

The library thus obtained is then biopanned using His-tagged S1 spike proteins (e.g., “SINOBIOLOGICAL INC. 40591-V08H”) resuspended in 400 μL of sterile u.p. water, in order to have a final concentration of 250 μg/mL.

200 μL of the aforementioned water, containing 50 μg of spike S1 proteins, is then taken and transferred into a microvial, for example a 1.5 mL microvial. 150 μL of sterile u.p. water and 350 μL of 2× Binding/Wash buffer are then added to the microvial, until a final volume of 700 μL of phage display library is reached. The 2× Binding/Wash buffer used is prepared for example with 11.98 gL⁻¹ di NaH₂PO₄ 100 mM) (e.g. “Fluka cat. 71496-1 kg lot. BCBC5685V”), 35.06 gL-1 NaCl (600 mM) (e.g., “Fluka CAT.S9888-1 kg lot. 12740”) and 0.02% Tween 20 (e.g., “SIGMA cat. P1379—250 mL lot. S8BE2460V”).

To functionalize the magnetic microspheres, 50 μL (2 mg) of His-tag magnetic microspheres are taken (for example, using a kit “Dynabeads His-tag isolation and pulldown—cat 10103D, 10104D Invitrogen”) and transferred to a sterile microvial, for example a 2 mL microvial, which is then placed on a magnet for 2 minutes. The spike S1 His-tagged protein previously diluted in 1× Binding/Wash buffer (700 μL) is then added to the microspheres and mixed. The microvial is then incubated on a wheel for 10 minutes at room temperature. After the incubation time has elapsed, the microvial is placed on a magnet for 2 minutes, and the supernatant is aspirated and discarded. Finally, four washes are carried out with 300 μL of 1× Binding/Wash buffer for 2 minutes, obtaining as a final result the microsphere/spike S1 complexes.

In order to select the phages against the spike S1 protein of the SARS-CoV-2 virus, the 700 μL of the previously obtained pre-adsorbed phage display library are mixed with the aforementioned microsphere/Spike S1 complexes and incubated in a wheel for 30 minutes at room temperature, obtaining microsphere/Spike S1/phage complexes. Then, the microvial containing the aforementioned complexes of microspheres/Spike S1/phages is placed on magnet for 2 minutes. The supernatant containing the remainder of the phage library not bound to the spike S1 protein is removed from the microvial and transferred to a new tube and stored at −80° C. On the microsphere/Spike S1/phage complexes remaining in the microvial, four washes are carried out with 300 μL of 1× Binding/Wash buffer, placing the microvial on the wheel for 5 minutes and on the magnet for 2 minutes at each wash and discarding the supernatant.

After the last wash of the microsphere/Spike S1/phage complexes, the phages are eluted in 200 μL of Glycine-BSA buffer (e.g., Glycine HCl “SIGMA cat. G8898-1 kg lot. 055k0188” 22.3 g/L and BSA “Applichem cat. A6588-0100 Lot. 5Y009437” 0.1 g/L) at pH 2.2 and incubated at room temperature for 20 minutes.

In order to allow the release of any phages still adhering to the microspheres with greater greed, the solution is preferably sonicated for 10 minutes at 20 kHz in an ice bath. The microspheres are then collected on a magnet for 5 minutes and the supernatant containing the phages is recovered. Finally, the eluate is neutralized with 150 μL of Tris-HCl buffer (1 M) at pH 9.1.

The pool of eluted phages obtained by the method exemplified above has the following titration values:

$$Total library title = 1 \times {10}^{13} TU / mL$$

$$Number of phages in 5 \times {10}^{10} TU / mL ( input )$$

$$Number of phages in 1 μL = 1 \times {10}^{10} TU / mL$$

$$Number of target - bound phages ( Spike S 1 proteins ) after selection = 5 \times {10}^{4} TU / mL$$

$$Yield = Number of phages linked to the target / input = 5 \times {10}^{4} / 5 \times {10}^{10} = 1 \times {10}^{- 6}$$

The pool of eluted phages obtained is then amplified, in order to increase the phage population from the selection.

In a first step of amplification, E. coli TG1 cells are incubated in lysogeny broth (LB) at 37° C. under agitation until an OD600 optical density of 0.8 is reached. Subsequently, 800 μL of E. coli TG1 are infected with 200 μL of suspension of the eluted phages from the selection and incubated at 37° C. for 15 minutes under static conditions and subsequently for 20 minutes under light agitation. An aliquot of 1 mL of this suspension of infected cells is inoculated in a 150 mm plate of LA+Ampicillin+Glucose medium and incubated at 37° C. for 16 h, in order to obtain E. coli TG1 cells infected with phages. A carpet of the aforementioned cells is then obtained on which 7 mL of LB is poured with 5 μL of ampicillin (from the 2000× stock solution) and 2.5 mL of 80% glycerol. The cells are then recovered with a spatula (scraping), transferred into 20 mL tubes and stored at −20° C. divided into aliquots.

In a second amplification step, 10 μL of cell suspension from the scraping is inoculated into 2 mL of LA+Ampicillin medium and incubated at 37° C. under agitation to an OD600 optical density of 0.4. An aliquot of 500 μL of this suspension is dispensed into a tube and 1 μL of phage helper M13K07 (1011 phages/mL) is added so as to have a final concentration of 109 phages/mL. The sample is incubated at 37° C. in static for 15 minutes and then for 20 minutes under stirring at 250 rpm. Infected cells are diluted in 1×PBS solution. Subsequently, 100 μL of the 10⁻³ and 10⁻⁴ dilutions are spatulated in LA+Ampicillin+IPTG+XGAL medium and incubated for 24 hours at 37° C. until blue colonies are obtained.

Approximately fifty random phages are then isolated and each phage chosen is labeled with S and a serial number, except one called S-α. Each colony is amplified and subsequently tested in ELISA to find the most reactive phage against the Spike S1 protein of the SARS-CoV-2 virus.

SARS-CoV-2 spike S1 protein was adsorbed overnight on a microtiter plate in carbonate-bicarbonate buffer. For the preparation of the CO₃²⁻/HCO₃⁻ buffer, 0.14 g of NaHCO₃ and 0.079 g of Na₂CO₃ were added to 50 mL of H₂O u.p. The solution is then filtered with 0.22 μm filter and 2 μL of Spike S1 protein are resuspended in 10 μL of CO₃²⁻/HCO₃⁻ buffer so as to use the Spike S1 protein at a final concentration of 5 μg/mL.

A wash is then performed with a wash buffer, manually shaking the plate for 3 minutes. 300 uL/well of blocking buffer (PBS+6% Milk+0.05% Tween 20) is then added and the plate is incubated at 37° C. for 2 hours. A wash is then performed with 300 uL/well wash buffer and 100 uL/well phage precipitate in TBS at the concentration of 1012 phages/mL is added and the plate is incubated at 37° C. for 1 hour. Five washes of 1 minute each are then performed with wash buffer, 100 uL/well of anti-M13-pVIII-HRP antibodies (batch aliquot: 9547458 n. 27-9421-01) diluted 1:5000 are added and the plate is incubated at 37° C. for 1 hour. Ten washes of 1 minute each are then performed with 300 μL/well of wash buffer and 100 μL/well of TMB is added. The plate is then incubated in the dark for 30 minutes and monitored to see the onset of phage staining; the reaction is blocked with 100 μL/well H₂SO₄. Finally, the absorbance (Spike ads) is measured at 450 nm using a microplate reader (e.g. a “Multiskan” reader). Non-functionalized microspheres were used as a control (K-).

The results obtained are shown in the following table:

| phage | Spike ads | control (K—) |
|---|---|---|
| S17 | 0.185 | 0.060 |
| S29 | 0.235 | 0.057 |
| S30 | 2.674 | 0.074 |
| S31 | 0.133 | 0.055 |
| S32 | 1.233 | 0.066 |
| S33 | 0.058 | 0.050 |
| S34 | 1.838 | 0.105 |
| S35 | 1.812 | 0.076 |
| S3 | 2.707 | 0.177 |
| S1-6 | 1.268 | 0.725 |
| S27 | 2.552 | 0.135 |
| S36 | 2.259 | 0.139 |
| S37 | 0.117 | 0.063 |
| S38 | 0.314 | 0.061 |
| S39 | 0.067 | 0.057 |
| S42 | 1.551 | 0.073 |
| S43 | 2.405 | 0.076 |
| S-α | 1.198 | 0.050 |

Absorbance values shown in bold in the previous table highlight the positive response in ELISA indicating that phages S30, S34, S35, S3, S1-6, S27, S36, S42, S43 and S-α are recognizing the spike S1 protein.

In addition, to evaluate the ability to recognize the most reactive phages not only towards the purified Spike S1 protein but towards the entire SARS-CoV-2 virions, an ELISA procedure was carried out in the manner described above, using the SARS-CoV-2 virus (for example, “amplirum total SARS CoV-2 control SWAB”) adsorbed on a microtiter plate as a target. Non-functionalized microspheres were used as a control (K-).

The results obtained are shown in the following table:

| phage | Virus ads | control (K—) |
|---|---|---|
| S1-6 | 3.83 | 0.152 |
| S11 | 0.770 | 0.155 |
| S31 | 0.218 | 0.139 |
| S33 | 0.184 | 0.069 |
| S34 | 0.705 | 0.166 |
| S35 | 0.094 | 0.063 |
| S36 | 0.084 | 0.058 |
| S42 | 0.065 | 0.058 |
| S43 | 0.105 | 0.057 |
| S-α | 3.59 | 0.2 |

The absorbance values shown in bold in the previous table highlight the positive response in ELISA indicating that S1-6, S11, S34 and S-α phages are recognizing the SARS-CoV-2 virus.

The DNA of S1-6, S11, S34 and S-α phages was amplified by PCR technique and then sequenced to identify the coding sequence for the p8 protein fusion peptide, capable of binding to the spike S1 protein.

The reaction mixture used for PCR amplification was: 21.25 μL of sterile PCR H2O, 10 μL of Buffer, 5 μL of each E24 primers (5′GCTACCCTCGTTCCGATGCTGTC 3′)-40 RE (5′GTTTTCCCAGTCACGAC 3′). The mixture was denatured in a thermocycler for 10 minutes at 95° C., and then 0.25 μL of my TAQ was added. Each sample was subjected to the following PCR cycles: 4 minutes at 94° C., 30 cycles of: 30 seconds at 94° C., 30 seconds at 52° C., 30 seconds at 72° C., 7 minutes at 72° C. At the end of the process, 35 μL were used for DNA purification and sequencing.

The sequencing results were as follows:
S1-6, S11 and S34 phages showed a nucleotide sequence corresponding to EFTSKAR;
S-α phage displayed a functional coding sequence motif for the FHKGGYEKTWKLGD peptide.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are to be regarded as a departure from the disclosure, and all such modifications are intended to be included within the scope of the disclosure.

1. A phage for the specific capture of the SARS-CoV-2 virus, said phage being an M13 phage engineered to display on the P8 protein of its coat either FHKGGYEKTWKLGD sequence peptides or EFTSKAR sequence peptides, said peptides having specific affinity for the Spike S1 protein of the SARS-CoV-2 virus.

2. A kit for capturing SARS-CoV-2 virus, said kit comprising a surface either functionalized with capture phages engineered to show on the P8 protein of their coat FHKGGYEKTWKLGD sequence peptides or EFTSKAR sequence peptides, or functionalized with FHKGGYEKTWKLGD peptides or EFTSKAR peptides.

3. The kit according to claim 2, wherein said surface is chosen from magnetic microspheres, metal electrodes, semiconductors or polymers.

4. The kit according to claim 3, wherein said magnetic microspheres have a diameter between 0.5 μm and 2.7 μm.

5. A method for detecting SARS-CoV-2 virus comprising the steps of:
providing a sample containing the SARS-CoV-2 virus,
capturing the SARS-CoV-2 virus either by means of capture phages engineered so as to show on the P8 protein of their coat FHKGGYEKTWKLGD sequence peptides or EFTSKAR sequence peptides, or by means of FHKGGYEKTWKLGD peptides or EFTSKAR peptides, each of said capture phages or peptides being immobilized on a surface,
isolating the SARS-CoV-2 virus by washing procedures, and
detecting, by transduction, the capture of the SARS-CoV-2 virus by said capture phages or said peptides.

6. The method according to claim 5, wherein said transduction is an optical or electrical or electrochemical or electrochemiluminescent transduction.

7. The method according to claim 5, wherein said capture phages are immobilized on magnetic microspheres and said step of isolating the SARS-CoV-2 virus by washing procedures comprises capturing the magnetic microspheres by means of a magnetic device.


SEQUENCE LISTING (4 sequences)

SEQ ID NO: 1 - protein, 14 aa, Inovirus M13
  source 1..14: note = Phage M13
FHKGGYEKTWKLGD

SEQ ID NO: 2 - protein, 7 aa, Inovirus M13
  source 1..7: note = Phage M13
EFTSKAR

SEQ ID NO: 3-4 - skipped (2 entries)